(19) United States
(12) Patent Application Publication
(10) Pub. No.: US 2011/0275802 A1
(43) Pub. Date: Nov. 10, 2011

(54) METHOD FOR MODIFYING DIISOCYANATES

(75) Inventors: Frank Richter, Leverkusen (DE); Reinhard Halpaap, Odenthal (DE)

(73) Assignee: BAYER MATERIALSCIENCE AG, LEVERKUSEN (DE)

(21) Appl. No.: 13/132,342

(22) PCT Filed: Nov. 26, 2009
(86) PCT No.: PCT/EP2009/008410

§ 371 Date: Jun. 2, 2011

(30) Foreign Application Priority Data
Dec. 3, 2008 (EP) 08170623.6

(51) Int. Cl. C07D 273/04; C07C 265/14; C07D 263/18; C07C 263/16; C07D 251/34

(52) U.S. Cl. 544/66; 560/351; 544/222; 560/334; 548/225; 544/68; 560/330

(57) ABSTRACT

The present invention relates to a process for modifying diisocyanates for preparing polyisocyanates, where the diisocyanate is reacted using a catalyst, wherein the diisocyanate used is obtained by quenching a gaseous reaction mixture in the phosgenation of diamines in the gas phase, wherein the gaseous reaction mixture comprises at least a diisocyanate, phosgene and hydrogen chloride, wherein a quenching liquid is sprayed into the gas mixture which flows continuously from a reaction zone into a downstream quenching zone, and wherein the quenching liquid is sprayed in by at least two spray nozzles which are arranged at the inlet of the quenching zone at equal intervals along the circumference of the quenching zone.

The present invention relates to a process for modifying diisocyanates for preparing polyisocyanates using a catalyst, where the diisocyanates used are obtained by a specific production process. The invention further relates to modified polyisocyanates prepared from abovementioned polyisocyanates and their use.

The production of 2-component polyurethane systems for paints and varnishes and other coatings usually proceeds from polyisocyanates based on diisocyanates together with suitable polyols. Preference is given to using aliphatic diisocyanates for high-quality surface coatings. Owing to the vapor pressure of the diisocyanates, it is important for the monomer content in the formulation of the polyurethane system to be as low as possible. For this reason, the aliphatic diisocyanates are generally modified; dimerizations and trimerizations are important types of reaction for this purpose. An overview of such reactions may be found, for example, in Journal far Praktische Chemie/Chemiker-Zeitung 1994, volume 336, pages 185 to 200.

The preparation of diisocyanates by phosgenation of diamines by-produces chlorine-containing compounds which can be summarized under the overall parameter of hydrolyzable chlorine (HC, HC content) are obtained as by-products. The term hydrolyzable chlorine is used for compounds which react with water to form hydrogen chloride or chloride ions, for example carbamoyl chlorides.

The reactions for modifying diisocyanates for the preparation of polyisocyanates of the dimer and trimer type are frequently carried out in the presence of a homogeneous catalyst, for example a phosphane. Thus, DE 103 54 544 A1 discloses the use of specific cycloalkylphosphanes as catalysts for isocyanate dimerization (uretdione formation) and a process for preparing polyisocyanates having a high content of uretdione groups.

It has been found in the past that diisocyanates which have a high content of hydrolyzable chlorine used for such modification reactions can lead to deactivation of the catalyst, for which reason there have been many attempts to prepare polyisocyanates which are very low in or free of HC and use these in the modification (on this subject see EP 1046637, EP 1053998).

Disadvantages of this procedure are firstly the increased costs associated with the additional process step of lowering the HC and secondly it has been found when using diisocyanates prepared by a phosgene-free process that a certain proportion of HC components which are absent as a result of the method in these process products actually have positive effects on, inter alia, the storage and color stability of the diisocyanates, for which reason stabilizers frequently have to be added thereto subsequently (EP 0643042).

It would be desirable to have alternative processes for modifying diisocyanates, in which slower deactivation of the catalyst occurs.

The invention therefore proposes a process for modifying diisocyanates for preparing polyisocyanates, where the diisocyanate is reacted using a catalyst.

The process is characterized in that the diisocyanate used is obtained by quenching the gaseous reaction mixture in the phosgenation of diamines in the gas phase, where the gaseous reaction mixture comprises at least a diisocyanate, phosgene and hydrogen chloride, a quenching liquid is sprayed into the gas mixture which, flows continuously from a reaction zone into a downstream quenching zone and the quenching liquid is sprayed in by means of at least two spray nozzles which are arranged at the inlet of the quenching zone at equal intervals along the circumference of the quenching zone.

A modification according to the invention of diisocyanates involves an increase in the molecular weight of these isocyanates, with the increase in molecular weight preferably taking place without reaction with compounds which do not contain any isocyanate groups. Such modifications are, in particular, dimerizations (uretdione formation), trimerizations (isocyanurate and iminooxadiazinedione formation) and/or oligomerizations to form species having uniform but different structure types of the abovementioned type in the oligomer or macromolecule.

For the purposes of the present invention, polyisocyanates are isocyanates having two or more isocyanate groups in the molecule, i.e. an NCO functionality of ≧2, and containing at least 2 units of the monomeric diisocyanates. Aliphatic, cycloaliphatic or araliphatic diisocyanates are advantageously used in the preparation of polyisocyanates. In particular, it is possible to use aliphatic diisocyanates which can also be branched and/or contain cyclic radicals.

The choice of the catalyst is initially not subject to any restrictions as long as it is suitable for the desired modification reaction.

The advantage of the process of the invention is firstly that even catalysts which have a simple structure and are thus inexpensive can be used, even if they are less active and therefore require long reaction times. In addition, it has been observed in the process of the invention that less strongly pronounced deactivation of the catalyst occurs. As a result, longer reaction times with the same catalyst and more uniform reaction conditions can be achieved.

The catalyst can be introduced into the reaction mixture neat or dissolved in a solvent. In the case of very active catalysts, dilution is sometimes advantageous in order to suppress spontaneous overcrosslinking reactions at the point at which the catalyst is introduced; less active catalysts are advantageously used neat. The catalyst can, for example, be used in a proportion of from ≧0.0005 mol % to ≦5 mol %, preferably from ≧0.001 mol % to ≦3 mol %, based on the amount of diisocyanate used.

According to the invention, use is made of diisocyanates which have been obtained from a gas-phase phosgenation, with quenching liquid (cooling liquid) being sprayed in. Such processes are described, for example, in DE 102 45 701 A1 or EP 1 403 248 B1, which are fully incorporated by reference.

In this way of preparing the diisocyanates, desired rapid cooling of the gas mixture comprising a diisocyanate, hydrogen chloride and excess phosgene is achieved by the spraying-in of a suitable quenching liquid. When the gas mixture leaves the reactor it has, for example, a temperature in the range from ≧300° C. to ≦400° C. and is cooled by the quenching to, for example, ≦150° C. The contact time in which the cooling occurs can be from ≧0.2 second to ≦3 seconds.

The spraying of the liquid can be effected by means of conventional spray nozzles or by means of openings, for example slits or holes, at the outlet from the reaction zone and/or at the inlet into the quenching zone. If only two spray nozzles are provided, these are preferably arranged diametrically opposite one another. The spray nozzles can be single nozzles. However, it is advantageous to use nozzle heads having in each case at least two single nozzles, with single-fluid nozzles preferably being chosen.

A further aspect of this way of preparing the diisocyanates is that the quenching liquid is sprayed into the gas stream in such a way that the hot reaction gas does not contact the relatively cold surfaces of the quenching zone or of the nozzles and their feed lines. Only when the gas mixture has been cooled to the temperature range in which the respective diisocyanate is stable does it come into contact with the relatively cold walls of the quenching zone or other components.

The spray nozzles are preferably arranged, independently of one another, so that in each case the flow direction of the quenching liquid is at an angle of from ≧0° to ≦50°, in particular from ≧20° to ≦35°, to the flow direction of the gas mixture. The flow direction of the gas mixture runs essentially along the axis of the cylindrical reaction zone or the quenching zone. In the case of a vertical arrangement of a tube reactor, the gas flows from the top downward through the reaction zone and the downstream quenching zone. Analogously, the flow direction of the quenching liquid runs along the axis of the spray nozzle. The opening angle of the spray nozzle is, independently, preferably from ≧20° to ≦90°, particularly preferably from ≧30° to ≦60°. In a particularly preferred variant, the flow direction of all the nozzles arranged in one plane are at the same angle to the flow direction of the gas mixture and also have the same opening angle.

In one embodiment of the process, the modification of the diisocyanates comprises formation of polyisocyanates having uretdione, isocyanurate, iminooxadiazinedione, polyamide, carbodiimide, uretonimine, urethane, allophanate, urea, biuret, amide, acrylurea, imide, oxazolidone, oxime carbamate and/or oxadiazinetrione structures. Preference is here given to dimerization or trimerization of the diisocyanates, i.e. modification to form uretdione, isocyanurate and/or iminooxadiazinedione structures.

In a further embodiment of the process, the diisocyanate is selected from the group consisting of hexamethylene diisocyanate, isophorone diisocyanate, tolylene diisocyanate and/or dicyclohexylmethane diisocyanate. In their monomeric form, these diisocyanates have comparatively high vapor pressures and are therefore generally modified.

In a further embodiment of the process, the catalyst comprises a tertiary phosphane. In particular, the catalyst can comprise phosphanes of the formula R¹R²R³P, where R¹, R² and R³ are, independently of one another, identical or different linear aliphatic, branched aliphatic and cycloaliphatic C₁-C₃₀ radicals. Thus, R¹ can be a cycloaliphatic C₃-C₃₀ radical and R² and also R³ are each, independently of one another, a cycloaliphatic C₃-C₃₀ radical or a linear or branched aliphatic C₁-C₃₀ radical. The cycloaliphatic C₃-C₃₀ radicals R¹, R² and R³ can additionally and independently of one another also be substituted by one or more C₁-C₁₂-alkyl or -alkoxy groups. R¹ is preferably a cyclopropyl, cyclobutyl, cyclopentyl or cyclohexyl radical. This radical can also be substituted by one or more C₁-C₁₂-alkyl or -alkoxy groups.

Preference is also given to R² and R³ each being, independently of one another, a cyclopropyl, cyclobutyl, cyclopentyl or cyclohexyl radical or an aliphatic C₂-C₈ alkyl radical. The cyclic radicals can also be substituted by one or more C₁-C₁₂-alkyl or -alkoxy groups.

Examples of cycloalkylphosphanes which can be used are: cyclopentyldimethylphosphane, cyclopentyldiethylphosphane, cyclopentyldi-n-propylphosphane, cyclopentyldiisopropyl-phosphane, cyclopentyldibutylphosphane, where “butyl” can be all isomers, i.e. n-butyl, isobutyl, 2-butyl, tert-butyl and cyclobutyl, cyclopentyldihexylphosphane (all isomeric hexyl radicals), cyclopentyldioctylphosphane (all isomeric octyl radicals), dicyclopentylmethylphosphane, dicyclopentylethylphosphane, dicyclopentyl-n-propylphosphane, dicyclopentylisopropyl-phosphane, dicyclopentylbutylphosphane (all isomeric butyl radicals), dicyclopentylhexylphosphane (all isomeric hexyl radicals), dicyclopentyloctylphosphane (all isomeric octyl radicals), tricyclopentylphosphine, cyclohexyldimethylphosphane, cyclohexyldiethylphosphane, cyclohexyldi-n-propylphosphane, cyclohexyldiisopropylphosphane, cyclohexyldibutylphosphane (all isomeric butyl radicals), cyclohexyldihexylphosphane (all isomeric hexyl radicals), cyclohexyldioctylphosphane (all isomeric octyl radicals), dicyclohexylmethylphosphane, dicyclohexyletylphosphane, dicyclohexyl-n-propylphosphane, dicyclohexylisopropylphosphane, dicyclohexylbutylphosphane (all isomeric butyl radicals), dicyclohexylhexylphosphane (all isomeric hexyl radicals), dicyclohexyloctylphosphane (all isomeric octyl radicals) and tricyclohexylphosphine.

These can be used as modification catalysts individually, in any mixtures with one another or in mixtures with other primary, secondary and/or tertiary alkylphosphanes, arylalkylphosphanes and/or arylphosphanes.

In a further embodiment, the catalyst comprises dicyclopentyl-n-butylphosphane. This compound is readily available and has shown in experiments that the activity is maintained for a long time in the process of the invention. Furthermore, owing to its boiling point, it can be removed together with unreacted diisocyanate from the reaction mixture, so that no catalyst remains in the resin obtained.

In yet another embodiment, the catalyst comprises phosphanes of the formula R⁴R⁵R⁶P, where R⁴ is a bicyclic aliphatic C₆-C₃₀ radical and R⁵ and also R⁶ are each, independently of one another, a monocyclic or bicyclic aliphatic C₄-C₃₀ radical or a linear or branched aliphatic C₁-C₃₀ radical. The cycloaliphatic C₄-C₃₀ radicals of R⁴, R⁵ and R⁶ can, independently of one another, also be substituted by one or more C₁-C₁₂-alkyl or -alkoxy goups.

Preference is given here to compounds in which R⁴ is a norbornyl radical (2.2.1-bicycloheptyl radical) substituted by one or more C₁-C₁₂-alkyl groups and R⁵ can, if desired, be identical to R⁴ or to R⁶ and R⁶ is an aliphatic C₁-C₁₂-alkyl radical substituted by one or more C₁-C₈-alkyl groups.

Examples of phosphanes which can be used are: norbornyldimethylphosphane, norbornyldiethylphosphane, norbornyldi-n-propylphosphane, norbornyldiisopropylphosphane, norbornyldibutylphosphane, where “butyl” can be all isomers, i.e. n-butyl, isobutyl, 2-butyl, tert-butyl and cyclobutyl, norbornyldihexylphosphane (all isomeric hexyl radicals), norbornyldioctylphosphane (all isomeric octyl radicals), dinorbornylmethylphosphane, dinorbornylethylphosphane, dinorbornyl-n-propylphosphane, dinorbornylisopropylphosphane, dinorbornylbutylphosphane (all isomeric butyl radicals), dinorbornylhexylphosphane (all isomeric hexyl radicals), dinorbornyloctylphosphane (all isomeric octyl radicals), trinorbornylphosphane, dimethyl(1,7,7-trimethylbicyclo[2.2.1]hept-2-yl)phosphane, diethyl(1,7,7-trimethylbicyclo-[2.2.1]hept-2-yl)phosphane, di-n-propyl(1,7,7-trimethylbicyclo[2.2.1]hept-2-yl)phosphane, diisopropyl(1,7,7-trimethylbicyclo[2.2.1]hept-2-yl)phosphane, dibutyl(1,7,7-trimethylbicyclo-[2.2.1]hept-2-yl)phosphane (all isomeric butyl radicals), dihexyl(1,7,7-trimethylbicyclo[2.2.1]hept-2-yl)phosphane (all isomeric hexyl radicals), dioctyl(1,7,7-trimethylbicyclo[2.2.1]hept-2-yl)-phosphane (all isomeric octyl radicals), methylbis(1,7,7-trimethylbicyclo[2.2.1]hept-2-yl)-phosphane, ethylbis(1,7,7-trimethylbicyclo[2.2.1]hept-2-yl)phosphane, n-propylbis(1,7,7-trimethylbicyclo[2.2.1]hept-2-yl)phosphane, isopropylbis(1,7,7-trimethylbicyclo[2.2.1]hept-2-yl)phosphane, butylbis(1,7,7-trimethylbicyclo[2.2.1]hept-2-yl)phosphane (all isomeric butyl radicals), hexylbis(1,7,7-trimethylbicyclo[2.2.1]hept-2-yl)phosphane (all isomeric hexyl radicals), octylbis(1,7,7-trimethylbicyclo[2.2.1]hept-2-yl)phosphane (all isomeric octyl radicals), dimethyl(2,6,6-trimethylbicyclo[3.1.1]hept-3-yl)phosphane, diethyl(2,6,6-trimethylbicyclo[3.1.1]-hept-3-yl)phosphane, di-n-propyl(2,6,6-trimethylbicyclo[3.1.1]hept-3-yl)phosphane, diisopropyl-(2,6,6-trimethylbicyclo[3.1.1]hept-3-yl)phosphane, dibutyl(2,6,6-trimethylbicyclo[3.1.1]hept-3-yl)-phosphane (all isomeric butyl radicals), dihexyl(2,6,6-trimethylbicyclo[3.1.1]hept-3-yl)phosphane (all isomeric hexyl radicals), dioctyl(2,6,6-trimethylbicyclo[3.1.1]hept-3-yl)phosphane (all isomeric octyl radicals), methylbis(2,6,6-trimethylbicyclo[3.1.1]hept-3-yl)phosphane, ethylbis(2,6,6-trimethylbicyclo[3.1.1]hept-3-yl)phosphane, n-propylbis(2,6,6-trimethylbicyclo[3.1.1]hept-3-yl)phosphane, isopropylbis(2,6,6-trimethylbicyclo[3.1.1]hept-3-yl)phosphane, butylbis(2,6,6-trimethylbicyclo[3.1.1]hept-3-yl)phosphane (all isomeric butyl radicals), hexylbis(2,6,6-trimethylbicyclo[3.1.1]hept-3-yl)phosphane (all isomeric hexyl radicals) and octylbis(2,6,6-trimethylbicyclo[3.1.1]hept-3-yl)phosphane (all isomeric octyl radicals).

The abovementioned compounds can be used as modification catalyst either individually, in any mixtures with one another or in mixtures with other primary, secondary and/or tertiary alkylphosphanes, aralkylphosphanes and/or arylphosphanes.

In a further embodiment of the process, the modification of the diisocyanates is carried out in continuous operation. This means that at least one component of the reaction mixture is circulated, so that more than one reaction run can be carried out. The continuous operation is advantageously carried out with the unmodified diisocyanate being separated off together with the catalyst from the modified polyisocyanates. The mixture of unmodified diisocyanate and catalyst which has been not deactivated or activated to only a small extent compared to known processes as a result of the diisocyanates used in the process of the invention can be replenished with fresh unmodified diisocyanate and reacted again. In a preferred variant, unmodified diisocyanate and catalyst are distilled off, with the modified polyisocyanate remaining as bottom product.

In a further embodiment of the invention, the quenching liquid is selected from the group consisting of toluene, monochlorotoluene, xylene, monochloronaphthalene, monochlorobenzene and/or dichlorobenzene. Monochlorobenzene and ortho-dichlorobenzene are particularly suitable. It is also possible to use a solution of the product diisocyanate in one of these organic solvents. Here, the proportion of solvent is preferably from ≧40% by volume to ≦90% by volume. In general, the temperature of the quenching liquid is preferably from ≧100° C. to ≦170° C.

In a further embodiment of the process, the reaction zone and/or quenching zone have a cylindrical shape. This is easy to realize in terms of apparatus and no thermal inhomogeneities arise when the reaction mixture comes into contact with container walls before or after quenching. The reaction zone is preferably a tube reactor without internals. Furthermore, it is possible for the diameter of the quenching zone to be greater than the diameter of the reaction zone.

The present invention further provides modified polyisocyanates obtained by the process of the invention. Examples of modification reactions and of selected polyisocyanates have been given above.

The present invention additionally provides for the use of modified polyisocyanates according to the invention for the production of moldings, foamed moldings, surface coatings, coating compositions, adhesives, sealants and/or aggregates. In the polyisocyanates, the free, unmodified NCO groups present may optionally also be blocked. As blocking agents, it is possible to use, in particular, phenols (for example phenol, nonylphenol, cresol), oximes (for example butanone oxime, cyclohexanone oxime), lactams (for example s-caprolactam), secondary amines (for example diisopropylamine), pyrazoles (for example dimethylpyrazole), imidazoles, triazoles or malonic and acetic esters.

The polyisocyanates prepared by the process of the invention can, in particular, be used for producing one- and two-component polyurethane coating composition. They can optionally be present in mixtures with other diisocyanates or polyisocyanates such as diisocyanates or polyisocyanates containing biuret, urethane, allophanate, isocyanurate and/or iminooxadiazinedione groups. It is likewise possible to use the polyisocyanates of the invention based on optionally branched, linear aliphatic diisocyanates as reactive diluents for reducing the viscosity of polyisocyanate resins which have a high viscosity or are solid at room temperature.

The coating compositions mentioned can be applied in solution or as a melt and also, in the case of powder coatings, in solid faint to the article to be coated by methods such as brushing, rolling, casting, spraying, dipping, fluidized-bed processes or electrostatic spraying processes. Suitable substrates are, for example, materials such as metals, wood, plastics or ceramics.

The preparation of the diisocyanates used according to the invention will be described further with the aid of the following drawing. In the drawing:

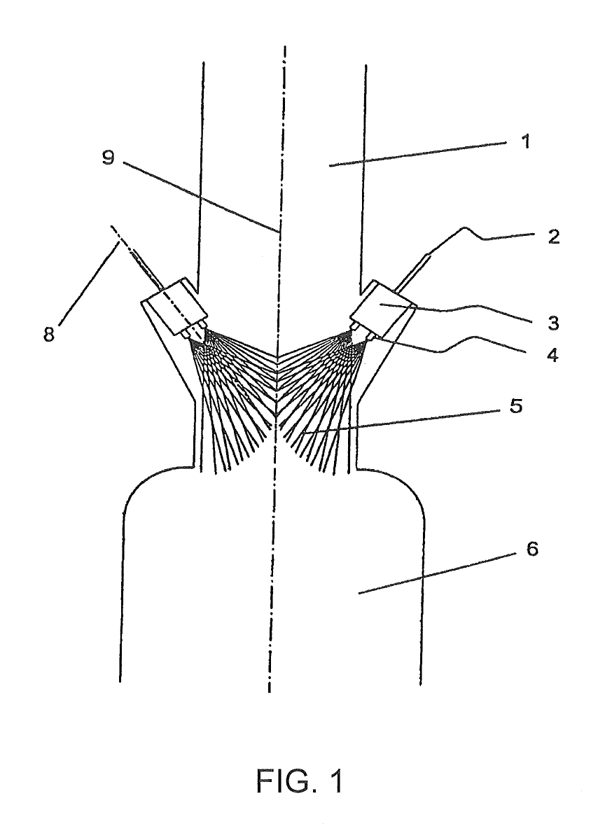

FIG. 1 shows a schematic cross section of a reaction zone and quenching zone.

FIG. 1 shows a cylindrical reaction zone 1 through which the gaseous mixture flows from the top downward along the broken line 9. On leaving the reaction zone 1, the gas mixture flows through a likewise cylindrical quenching zone 5. Two nozzle heads 3 each having two individual nozzles 4 are arranged diametrically opposite one another in the quenching zone 5. The nozzles 4 or the nozzle head 3 are arranged so that the flow direction of the quenching liquid (represented by the broken line 8) and that of the gas stream 9 are at an angle of from 0° to 50°, in particular from 20° to 35°, to one another and the hot gas mixture therefore does not come into contact with the colder nozzles or nozzle head. In the quenching zone 5, the reaction gas is cooled by vaporization of the atomized liquid. The remaining liquid and the cooled reaction gas go into the liquid collection vessel 6 underneath, which simultaneously serves as pump reservoir and as separation apparatus for gas and liquid.

The present invention will be illustrated further with the aid of the examples according to the invention below and the comparative example. Here, to illustrate the system, HDI has been selected as diisocyanate to be modified and tertiary phosphane has been selected as catalyst, which does not imply that the present invention is restricted to this diisocyanate or this type of catalyst.

In the examples, all percentages are, unless indicated otherwise, by weight. The reactions were carried out using freshly degassed hexamethylene diisocyanate (HDI) as starting material. Here, the term “degassed” means that the HDI used is freed of dissolved gases, in particular CO₂, by stirring under reduced pressure (<1 mbar) for at least 30 minutes and subsequently blanketed with nitrogen immediately before the catalytic reaction.

All reactions were carried out under a dry nitrogen atmosphere. The chemicals and catalysts described in the examples were procured from Bayer (HDI) or Cytec (phosphane) and were used without further purification.

### Example 1

### Example According to the Invention

1050 g of HDI prepared as described in DE 102 45 704 A1 were placed in a double-walled flange vessel provided with a stirrer, thermometer and a reflux condenser connected to an inert gas system (nitrogen/vacuum) and maintained at 30° C. by means of an external circuit and the HDI was degassed. After admission of nitrogen, 12 g of dicyclopentyl-n-butylphosphane were added and the mixture was stirred at 30° C. for about 24 hours. The reaction mixture was subsequently worked up without prior deactivation of the catalyst. The work-up was carried out by vacuum distillation (0.08 mbar) in a thin film evaporator of the short path evaporator type (SPEv; temperature of the heating medium: 150° C.) with upstream preevaporator (PEv, temperature of the heating medium: 120° C.) with unreacted monomer being separated off together with the active catalyst as distillate and the polyisocyanate resin being separated off as bottom product.

This reaction mixture was the starting mixture having the experiment number 1-0 as per table 1 below.

The distillate containing the active catalyst was introduced into a second stirred flange apparatus constructed identically to that described above and immediately after the end of the distillation, made up to the initial amount (1050 g) with freshly degassed (HDI). The mixture was subsequently stirred again at 30° C. for about 24 hours and worked up as described above. This reaction mixture has the experiment number 1-A in table 1.

This procedure was repeated a total of 24 times (up to experiment number 1-X in table 1), with experiments 1-E, 1-J, 1-0 and 1-T running for about 72 hours in order to achieve higher resin yields and only then being worked up.

Over the course of the trial carried out over a number of weeks, an only slow decrease in the catalytic activity was observed, with the decreasing resin yield being employed as measure of the decrease in the catalytic activity. The relative activity of the catalyst was calculated by dividing the resin yield obtained in the reaction batch by 1050 g, i.e. the weight of the HDI which was used and to be modified, and then forming the ratio to the resin yield in the starting mixture (defined as 100% relative activity).

TABLE 1
(example according to the invention)

| Experiment number | Relative activity [%] |
|---|---|
| 1-0 | 100 |
| 1-A | 100 |
| 1-B | 97 |
| 1-C | 97 |
| 1-D | 99 |
| 1-E | 80 |
| 1-F | 100 |
| 1-G | 79 |
| 1-H | 76 |
| 1-I | 74 |
| 1-J | 64 |
| 1-K | 92 |
| 1-L | 81 |
| 1-M | 82 |
| 1-N | 75 |
| 1-O | 56 |
| 1-P | 80 |
| 1-Q | 68 |
| 1-R | 66 |
| 1-S | 66 |
| 1-T | 55 |
| 1-U | 71 |
| 1-V | 74 |
| 1-W | 76 |
| 1-X | 71 |

### Example 2

### Comparative Example

The procedure of example 1 was repeated using HDI which had not been prepared as described in DE 102 45 704 A1. The HDI was obtained by a phosgenation process as described in EP 02 89 840 and had the same content of hydrolyzable chlorine (HC content) as the HDI of example 1, namely 22 ppm. The results are summarized in table 2. As can be seen, the activity of the catalyst here decreases significantly more quickly with continuing recycling of the monomer than in the example according to the invention.

TABLE 2
(comparative example)

| Experiment number | Relative activity [%] |
|---|---|
| 2-0 | 100 |
| 2-A | 98 |
| 2-B | 97 |
| 2-C | 96 |
| 2-D | 94 |
| 2-E | 78 |
| 2-F | 88 |
| 2-G | 75 |
| 2-H | 72 |
| 2-I | 70 |
| 2-J | 59 |
| 2-K | 75 |
| 2-L | 69 |
| 2-M | 65 |
| 2-N | 61 |
| 2-O | 49 |
| 2-P | 55 |
| 2-Q | 51 |
| 2-R | 45 |
| 2-S | 39 |
| 2-T | 28 |
| 2-U | 33 |
| 2-V | 31 |
| 2-W | 26 |
| 2-X | 24 |

1.-10. (canceled)

11. A process for modifying diisocyanates for preparing polyisocyanates, where the diisocyanate is reacted using a catalyst, wherein the diisocyanate used is obtained by quenching a gaseous reaction mixture in the phosgenation of diamines in the gas phase,
wherein the gaseous reaction mixture comprises at least a diisocyanate, phosgene and hydrogen chloride,
wherein a quenching liquid is sprayed into the gas mixture which flows continuously from a reaction zone into a downstream quenching zone, and
wherein the quenching liquid is sprayed in by at least two spray nozzles which are arranged at the inlet of the quenching zone at equal intervals along the circumference of the quenching zone.

12. The process as claimed in claim 11, wherein the polyisocyanates formed comprise uretdione, isocyanurate, iminooxadiazinedione, polyamide, carbodiimide, uretonimine, urethane, allophanate, urea, biuret, amide, acrylurea, imide, oxazolidone, oxime carbamate and/or oxadiazinetrione structures.

13. The process as claimed in claim 11, wherein the diisocyanate is selected from the group consisting of hexamethylene diisocyanate, isophorone diisocyanate, tolylene diisocyanate and dicyclohexylmethane diisocyanate.

14. The process as claimed in claim 11, wherein the catalyst comprises a tertiary phosphane.

15. The process as claimed in claim 14, wherein the catalyst comprises dicyclopentyl-n-butylphosphane.

16. The process as claimed in claim 11, wherein the modification of the diisocyanates is carried out in continuous operation.

17. The process as claimed in claim 11, wherein the quenching liquid is selected from the group consisting of toluene, monochlorotoluene, xylene, monochloronaphthalene, monochlorobenzene and dichlorobenzene.

18. The process as claimed in claim 11, wherein the reaction zone and/or the quenching zone are cylindrical.

19. A modified polyisocyanate obtained by the process as claimed in claim 11.

20. A molding, foamed molding, surface coating, coating composition, adhesive, sealant and/or aggregate produced with the polyisocyanates as claimed in claim 19.
